(19) United States
(12) Patent Application Publication
(10) Pub. No.: US 2018/0056552 A1
(43) Pub. Date: Mar. 1, 2018

(54) New dipping former for producing elastic articles

(71) Applicant: Weng Kee Richard BOEY, PETALING JAYA (MY)

(72) Inventor: Weng Kee Richard BOEY, PETALING JAYA (MY)

(21) Appl. No.: 15/556,597

(22) PCT Filed: Mar. 9, 2016
(86) PCT No.: PCT/MY2016/000010

§ 371 Date: Sep. 7, 2017

(30) Foreign Application Priority Data
Mar. 18, 2015 (MY) PI2015000677

(51) Int. Cl. B29C 41/40; B29C 33/08; B29C 45/00; A61B 42/00; A41D 19/04; A41D 19/00; B05D 7/00

(52) U.S. Cl. CPC B29C 41/40; B29C 33/08; B29C 45/0053; B29L 2031/4864; A41D 19/04; A41D 19/0058; B05D 7/52; A61B 42/00

(57) ABSTRACT

A former assembly for the manufacture of dip product, the assembly comprising: a thermally conductive outer layer in the shape of said product, said outer layer arranged to receive a film of elastomer; a mounting for mounting said former assembly to a former holder for engagement with a conveyor chain; a heating medium within said outer layer, said heating medium in communication with an energy source for heating said medium; wherein said heating medium arranged to apply heat through said outer layer so as to cure said resin.

### FIELD OF THE INVENTION

The invention relates to the production of dipped products of elastomeric material. In particular, the invention relates to the means of drying and curing the elastomer to form the glove and other dipped products whilst it is on the former assembly.

### BACKGROUND

The production of elastomeric gloves and other dipped products involves a conveyor or batch system having a plurality of formers attached. The formers passes through various stages including a dipping stage where the glove formers are dipped within a liquid elastomeric resin, an oven stage for drying and curing the elastomeric film and an extended drying stage.

It is the oven stage to which the present invention is directed. The conventional oven comprises a heating source for elevating the space within the oven to a sufficient temperature to both heat the resin so as to cure the gloves, for example, and maintain the oven at a temperature to prevent “cold spots” developing that may affect the curing process. The length of the oven is determined as a function of the speed the conveyor and the time required to cure the resin. Maximizing the rate of production of the gloves requires the conveyor to be travelling at a high speed, and so the length of the oven, to ensure sufficient curing time, needs to be of considerable length, and consequently, requires heating of a considerable volume within the oven. It follows that the energy required to heat this volume is also considerable.

By volume, the gloves and other dipped products, represent an extremely small proportion of the space within an oven. Therefore, the actual energy used to cure the resin, as compared to the energy required to maintain the temperature of the oven is also extremely small. It is estimated that the energy required to heat the oven is in the range of 90 to 95% of the total heat used. That is, the actual energy to cure the resin is only about 5 to 10% of the total energy. The cost of the wasted energy represents a significant cost of the manufacture of the glove, which is ultimately dissipated to the environment.

It follows, therefore, that any reduction or saving in this wasted energy will have a direct effect on the cost of the glove manufacture, and so provide a significant commercial advantage.

### SUMMARY OF INVENTION

In a first aspect, the invention provides a former assembly for the manufacture of dip product, the assembly comprising: a thermally conductive outer layer in the shape of said product, said outer layer arranged to receive a film of elastomer; a mounting for mounting said former assembly to a former holder for engagement with a conveyor chain; a heating medium within said outer layer, said heating medium in communication with an energy source for heating said medium; wherein said heating medium arranged to apply heat through said outer layer so as to cure said resin.

By providing a separate heat source within each former, the ovens can be discarded from the conveyor line leading to two substantial advantages. First and foremost, the energy required to cure the glove using the system according to the present invention is only 5 to 10% of that of a prior art system. Further, the infrastructure cost in removing the ovens may lead to a substantial reduction in the amortized cost of the manufacturing plant.

Further, the length of the conveyor line may also be substantially reduced, as there is no specific length of conveyor required for the oven stage.

Further still, the gloves may be cured at a temperature controlled by the operator at a much higher efficiency than that compared to the prior art. Whereas the prior art systems can waste in a range 90% to 95% of the generated heat to the atmosphere, it is effectively impossible to optimize the amount of energy used to cure each glove. By using a system according to the present invention, the optimal amount of energy to cure each glove may be selected and so in addition to the gross savings from adopting the present invention compared to the prior art, the ability to fine tune the energy output may also be available.

In a second aspect the invention provides a method of manufacturing a former assembly comprising the steps of: forming a thermally conductive outer layer in the shape of a dip product, said outer layer having an inner surface, and an outer surface arranged to receive a film of elastomer; forming an electrically conductive layer on said inner surface; placing a pair of resistive patterned tracks on said electrically conductive layer; connecting said pair of resistive patterned tracks to an electrical power supply; electrically connecting said pair via an intermediate portion of said electrically conducting layer and, generating resistive heat from said intermediate portion for curing said elastomer.

In a third aspect the invention provides a former assembly for the manufacture of dip product, the assembly comprising: a thermally conductive outer layer in the shape of said product and having an inner and outer surface, said outer layer arranged to receive a film of elastomer; an electrically conductive layer on said inner surface; a pair of resistive patterned tracks on said electrically conductive layer and in communication with an electrical power supply; wherein said pair of resistive patterned tracks is arranged to permit selective heat distribution across said former assembly for curing said elastomer.

Relative to known methods, the present invention provides means for manufacturing a former assembly which is capable of generating resistive heat with minimal electrical conducting material mass. The significant reduction in material mass simplifies design and methods of manufacturing former assemblies. It follows that capital investment and long term operating cost may therefore be reduced.

In one embodiment, the placing step further includes the steps of varying one or more dimensions of said pair of resistive patterned tracks at pre-determined areas of said outer layer, and so varying a distribution of heat about said electrically conductive layer.

In alternative embodiments, said resistive patterned tracks are formed using electro-deposition.

The present invention provides a means for varying and fine tuning the distribution of heat about the electrically conductive layer for drying and curing the elastomeric film. It may be appreciated that the heat provided, as a result of electrical connection between the pair of resistive patterned tracks and the electrically conductive layer, may be transferred to the elastomer formed on the outer layer or outer shell so as to dry and cure the elastomer. Further, heat distributed within a former assembly may be varied by changing or varying the dimensions, such as thickness, width and length, of the resistive patterned tracks at pre-determined or targeted areas of the outer layer. For instance, a thin layer of the resistive patterned track may be placed at pre-determined area of the electrically conducting layer if lesser heat distribution is desired. Alternatively, a resistive patterned track will not be placed within a pre-determined area of the electrically conductive layer if no heat is desired. Further, additional pairs of resistive patterned tracks may be deposited at areas where a greater heat intensity and distribution are desired. It will be appreciated that the arrangement of resistive patterned tracks on electrically conductive layers may be customized according to requirements of an intended application.

The present invention is thus advantageous relative to the prior art because of the ability to fine tune the energy output provided for the manufacturing dip products. This may in turn translate to one or a combination of the following benefits:
reduces energy wastage resulting in gross capital and operational savings; and
indirectly permitting control over the pick-up rate, that being the amount of elastomer formed on the outer layer of the former assembly, and the curing rate of the elastomer.

### BRIEF DESCRIPTION OF DRAWINGS

It will be convenient to further describe the present invention with respect to the accompanying drawings that illustrate possible arrangements of the invention. Other arrangements of the invention are possible and consequently, the particularity of the accompanying drawings is not to be understood as superseding the generality of the preceding description of the invention.

FIGS. 1A and 1B are various views of a glove former shell assembly according to one embodiment of the present invention;

FIGS. 2A and 2B are various views of a disassembled glove former shell assembly according to a further embodiment of the present invention;

FIG. 3 is an isometric view of a glove former inner heater core assembly according to a still further embodiment of the present invention;

FIG. 4 is an isometric exploded view of the glove former assembly of FIG. 3;

FIGS. 5A and 5B are isometric views of glove manufacturing conveyor lines utilising electrification to energise the glove former according to various embodiments of the present invention;

FIG. 6 is a side view of internal layer arrangements of a glove former shell assembly, according to another embodiment of the present invention;

FIG. 7 is a side view of a glove former shell assembly, which incorporates the internal layer arrangements of FIG. 6;

FIG. 8 is a side view of internal layer arrangements of a glove former shell assembly, according to yet a further embodiment of the present invention.

FIGS. 9 and 10 are schematic block diagrams of methods of manufacturing a former assembly according to various embodiments of the present invention.

FIGS. 11A and 11B are various views of disassembled glove former shell assemblies manufactured according to the methods of manufacturing in FIGS. 9 and 10.

FIG. 12 is a heat distribution profile of a glove former shell assembly according to a further embodiment of the present invention.

In describing the preferred embodiments of the invention, specific terminology will be resorted to for the sake of clarity. However, it is not intended that the invention be limited to the specific terms so selected and it is to be understood that each specific term includes all technical equivalents that operate in a similar manner to accomplish a similar purpose. Combination of the various embodiments of the present invention as described herein may also be used depending on specific facility requirements.

### DETAILED DESCRIPTION

The following description will refer to the invention applied to the manufacture of gloves, for convenience. It will be recognised that the invention may be applied to any dip product, including condoms, probe sheaths and other such objects formed by moulds dipping into an elastomer bath. Accordingly, the reference to glove manufacture is not to be read as limiting the application of the invention.

The core invention involves providing a heating medium within a glove former assembly so as to transfer heat from the heating medium within the assembly to the elastomer formed on the outer layer, which may be an outer shell, of the former so as to dry and cure the glove. This is contrast to passing the glove former assembly through an oven heated to a temperature to not only cure the elastomeric glove film but also to maintain heat within the oven to a degree beyond that required for the resin curing.

In achieving the invention, one embodiment may include applying an electrically conductive coating to an injection molded thermo-plastic former, then forming patterned tracks in a cavity of the former in a 3 dimensional configuration so as to have an internally positioned electrically resistive heater.

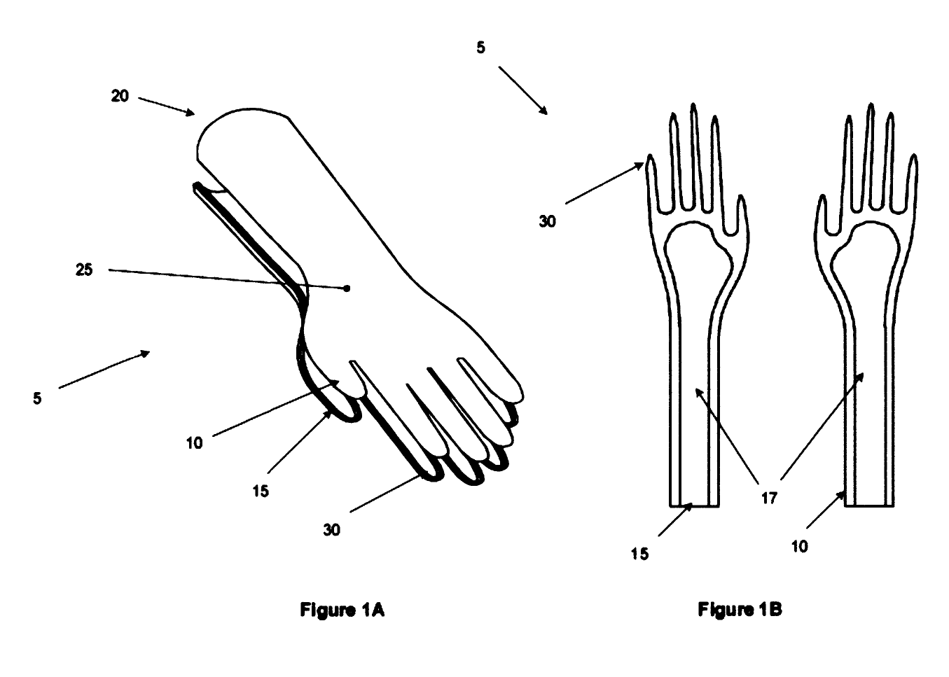

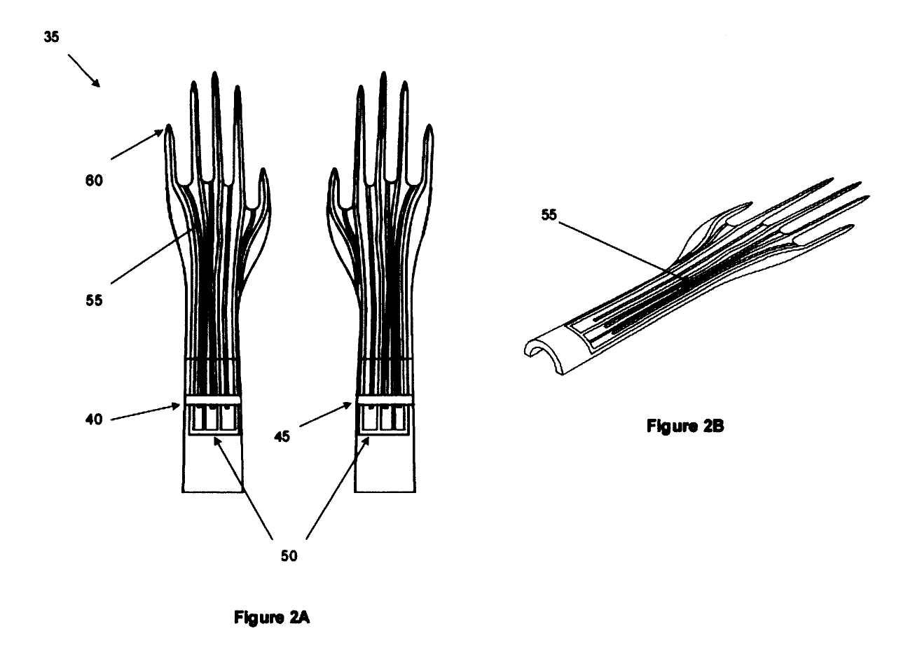

FIGS. 1A and 1B show one embodiment of the internal heating system for a glove former assembly 5. The assembly 5 includes a thermally conductive outer shell for receiving the elastomeric film. In this embodiment, the outer layer may comprise an outer shell in two halves 10, 15, which may be attached to enclose the heating medium within a void 17 of the outer shell 5. The two halves may be permanent sealed, such as through heat sealing or releasably engaged to provide maintenance to the internal heating medium. Heat for the heating medium (not shown) may be provided through a connection to the holder adjacent the cuff area 20. Heat is then generated so as to apply heat to a surface of the layer 25 in order to cure the elastomer on said surface 25. A characteristic of the internal heat includes ensuring sufficient heat is generated at the extremities 30 of the glove former to establish uniform curing.

Such heating sources will depend upon the heating medium, and may include a convective heat source such as a flowing hot liquid medium, for instance water, or a conductive heat source such as heating a gel resident within the outer shell. In this case, there may be a thermally conductive member intermediate the heating source and the gel to transfer heat directly. In either case, access to the inner portion of the outer layer/shell may be through the cuff 20. This may require a modification of the holder (not shown), such as by providing an annular rotatable ring, and passing the heating conduit through said ring. Other means of heating the glove former assembly may include various forms of electrical heating as will be described with reference to further embodiments.

The outer shell/layer 5 may be of thermally conductive materials, such as modified Polyphenylene Sulfide (PPS), modified Glass Reinforced Plastic (GRP) or a ceramic material sufficient to efficiently conduct heat from the heat source to the elastomeric coating the outer layer

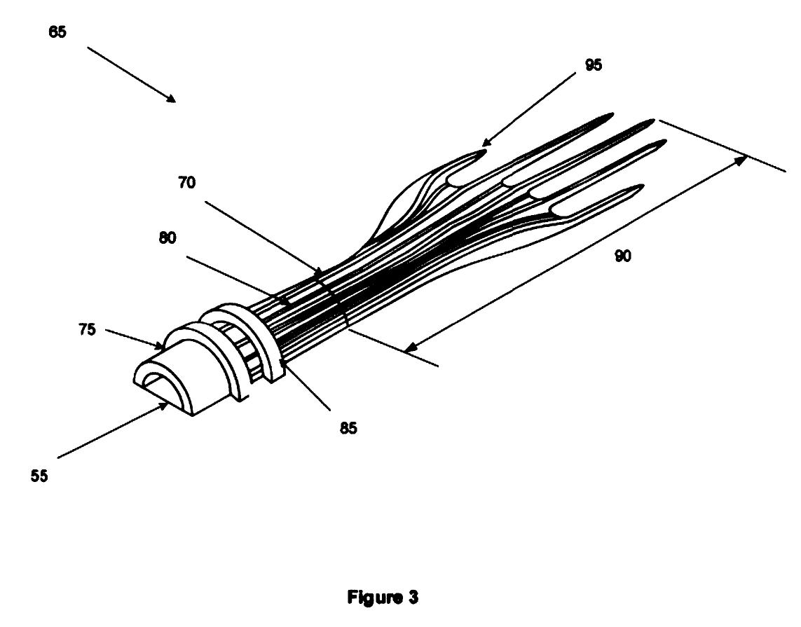

FIGS. 2A and 2B show one such electrical system whereby a glove former assembly 35 has been split in half to show a left side 40 and right side 45. The assembly shown in FIGS. 2A and 2B may be an internal core upon which an outer layer or shell (not shown) may be added, said outer layer arranged to receive the elastomer and upon which the glove may be formed. By way of example, the inner core of FIGS. 2A and 2B may substitute for other heating mediums by placing within the outer layer 5 of FIG. 1. It will also be appreciated that this configuration of busbars may be mounted on an internal surface of the outer shell rather than to a discrete inner core, and so have the “inner core” integral with the outer shell.

As can be seen, a busbar configuration 55, 60 is distributed throughout the inner core 35 with an isolating strip 50 for isolating the busbar arrays. In this embodiment, the busbars are arranged as elongate filaments positioned in parallel with alternating positive and negative strips. The busbars are connected at an extreme point (not shown) of the former. By applying a current from an electrical power supply (not shown) to the busbars 55, 60, resistive heat is generated, with the greater the concentration of filaments, the higher the heat generated. The resistive heat is then communicated to the outer layer (not shown) so as to heat the glove. An advantage of this arrangement is the ability to place busbars 60 at the extremities through the fingers of the glove, because of the fine arrangement of the filaments, so as to ensure uniform curing of the glove. The busbars may be placed and adhered to the surface of the core. Alternatively, they may be added as a composite within an injected moulded section. A still further alternative may include a damascene construction, whereby the busbars are placed within corresponding recesses within the internal core surface.

In one embodiment, the electrical power supply may operate in the range 5 to 50 volts, and possibly in the narrower range 10 to 30 volts. In so doing, the design system may operate at a low voltage high current. Accordingly, the electrical output is arranged to be at a safe level for human interaction.

In a still further embodiment, the heating medium may be integral with the outer layer/outer shell. For convenience, in this arrangement, the busbars may be placed on an inside face of the former. In this case, the medium may include a substantial portion of the thickness of the former. In certain circumstances, the outer shell and medium may be visually indistinguishable, with the external directed heat transfer from the busbars to the elastomer on an outer surface of the outer shell indicating the notional position of the medium.

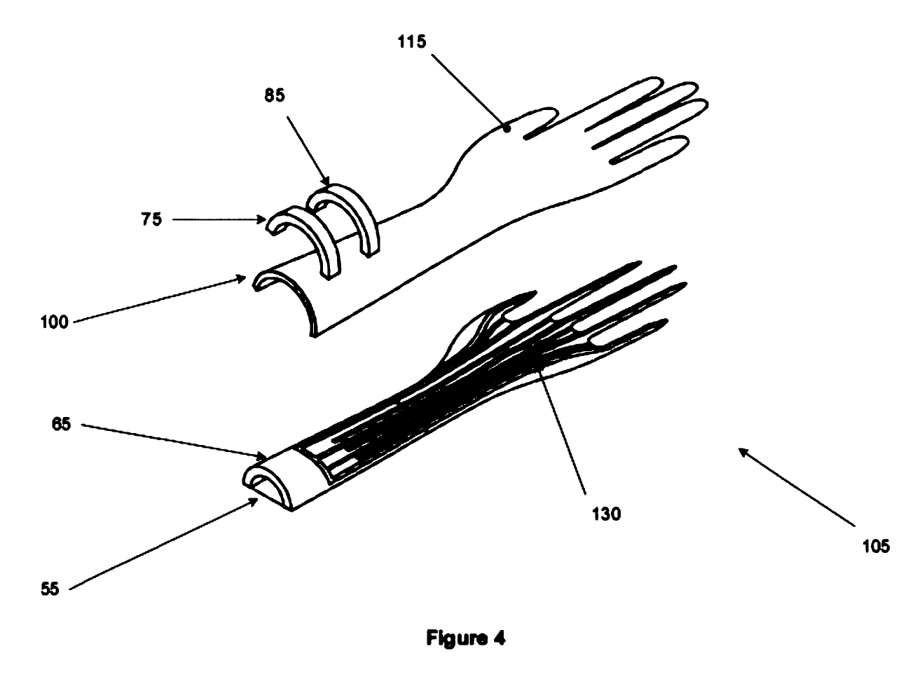

FIGS. 3 and 4 show a further embodiment of the present invention whereby again the inner core 65 includes busbars 70, 80 distributed throughout the core. However, in this embodiment a portion 90 of the inner core includes an electrically conductive layer, for instance graphite. To this end, the busbar configuration includes two discrete arrays 70, 80, with the electrical connection between the arrays 70, 80 provided through the electrically conductive layer. Alternatively, the layer may be a heat generating graphite layer or a conductive polymer layer, such as polyacetylene, polypyrrole, and polyaniline. The advantage of this embodiment is to provide resistive heat uniformly about the inner core and not merely proximate to the busbars. This has particular advantage in providing uniformity to the curing process. In particular, the glove extremities 95 such as the fingers require the transfer of sufficient heat to provide a particularly high quality finish to the gloves.

Whereas FIG. 3 shows detail of one half of the inner core 65, FIG. 4 shows an exploded view of one half of the glove former assembly 105, comprising the thermally conductive outer layer 115 and the inner core 65 of FIG. 3. The inner core fits within the outer layer 115 and may be sealed permanently through heat sealing, or adhesion, or may be selectively disassembled through screws or similar.

Whilst FIG. 3 shows the polarity rings 75, 85 attached directly to the inner core, it may be convenient to extend the outer layer for the full length of the inner core, and so have the polarity rings 75, 85 engaged at an end portion 100 of the outer layer, with electrical penetrations through the outer layer to connect with the respective busbars 70, 80.

It will be appreciated that whilst the embodiment of FIGS. 3 and 4 may provide a high quality finish compared to those embodiments of FIGS. 1 and 2, the costs of constructing such a former will correspondingly be higher. It would therefore be within the control of the manufacturer to balance infrastructure costs with the quality level required of the finished product. Nevertheless, all embodiments involving the internal generation of heat from the glove former assembly fall within the scope of the present invention.

The busbars 70, 80 are connected to polarity rings 75, 85, for instance with a negative polarity ring 75 connected to the negative busbars 70 which are returned through the electrically conductive layer 130 to the positive polarity ring 85 via the positive busbars 80.

Between halves of the inner core is provided a positive polarity common ring main plate 55 being a plate of conductive metal such as aluminium, copper, steel etc. The electrically conductive plate 55 is to connect the positive polarities.

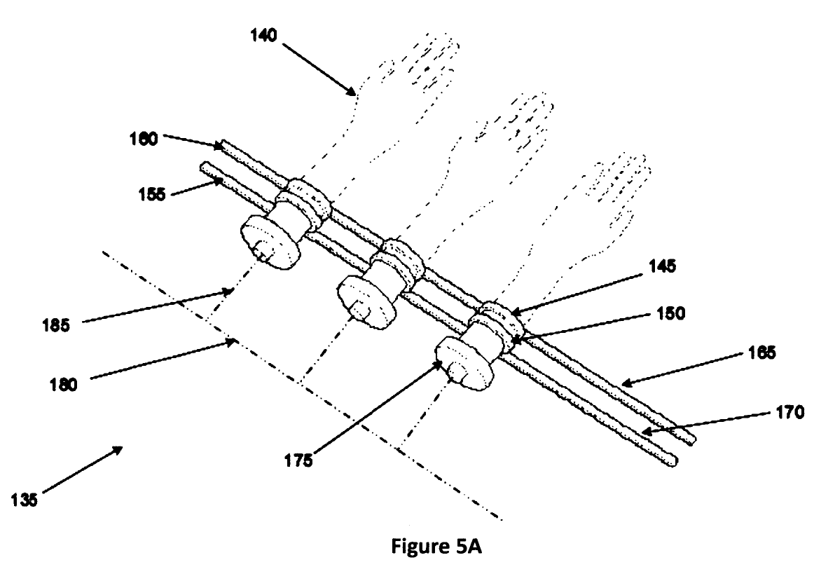

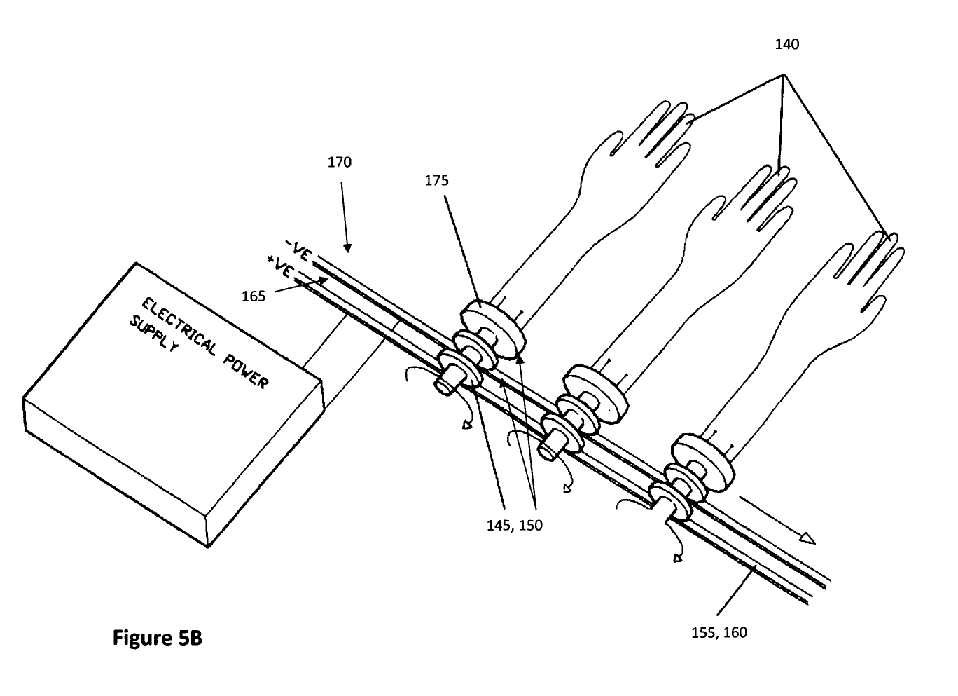

FIG. 5 shows the glove former assembly in context whereby a plurality of glove former assemblies 140 are mounted to holders 175 which are mounted 185 to a conveyor 180 (not shown) so as to form a glove manufacturing conveyor system 135.

Here the internal core (not shown) receives a power supply through the polarity rings 145, 150 through electrically conductive tracks 155, 160 specifically a negative track 170 and a positive track 165. The polarity rings remain in contact with the tracks, and so act to rotate the glove formers in the same way the former holder at different stages within the conveyor system engage a separate track in order to rotate the holders. Thus, the means of providing a power supply to the inner core is seamlessly introduced into the conventional design for a glove system to rotate the gloves as required.

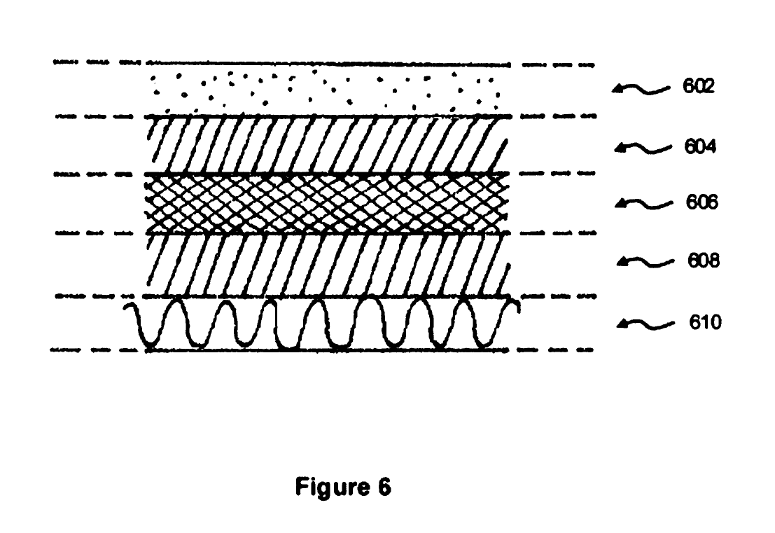

FIG. 6 is a side view of internal layer arrangements 602-610 of a glove former shell assembly, according to another embodiment of the present invention. Specifically, describing in a top down order of internal layer arrangements 602-610 as depicted in FIG. 6, the internal layer arrangements 602-610 include an outer layer 602, a first busbar layer 604, a heat generating layer 606, a second busbar layer 608, and a heat insulating layer 610, said heat generating layer intermediate the first and second busbar layers. The outer layer 602 is formed from a thin layer of thermoplastic material (e.g. modified PPS) which is substantially chemically inert and heat resistant, since the outer layer 602 will interact with and be exposed to the elastomer bath. Immediately underneath the outer layer 602 is the first busbar layer 604, which is an electrically and thermally conductive integral layer (e.g. thin aluminium shell), configured to function as an electrical busbar layer and also as a heat spreader. In this instance, the first busbar layer 604 is electrically arranged with a positive polarity (i.e. “+VE”).

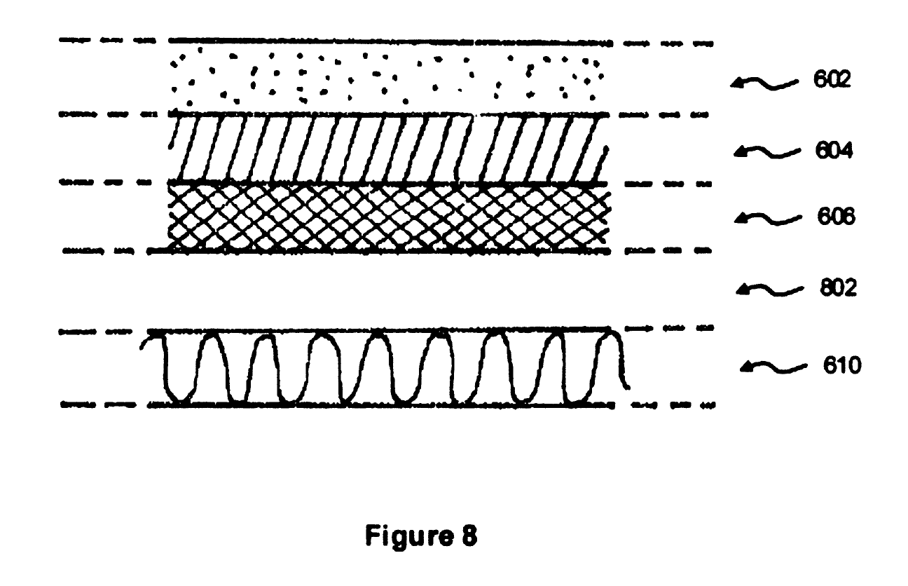

It will be appreciated that the first and/or second busbar layers may be filament arranged layers sufficient to be electrically or thermally conductive. Alternatively, and as shown in FIGS. 6 and 8, said layers may be continuous.

Further, it will be appreciated that the heat generating layer may be continuous, in that it substantial coats the former. Alternatively, the heat generating layer may be a discontinuous layer, such as an array of discrete “patches” placed about the former.

Disposed immediately underneath the first busbar layer 604 is the heat generating layer 606, which is a mixture of carbon nanotube graphite and ceramic power that exhibits Positive Temperature Coefficient (PTC) properties. An example of the ceramic powder, but not limited only to the described, is Barium Titanate, which is mixed in a ratio of approximately between 2% to 40%, preferably 3% to 35% and most preferably 5% to 20% (calculated by weight to weight basis of fine Barium Titanate powder) with the carbon nanotube graphite, and the resulting mixture is then bended and heat cured to form the heat generating layer 606. It is to be appreciated that the heat generating layer 606 is a solid integral layer and is about 1-2 mm thick. In addition, the heat generating layer 606 is arranged intermediate between the first and second busbar layers 604, 608, as will be appreciated from FIG. 6.

Further, immediately underneath the heat generating layer 606 is the second busbar layer 608, which is materially and structurally similar to the first bus bar layer 604, except that the second busbar layer 608 is electrically arranged with a negative polarity (i.e. “−VE”). The first and second busbar layers 604, 608 are collectively electrically connected to an electrical power source (not shown) in order to provide the necessary electrically energy to energise the heat generating layer 606 for generating heat. In other words, the heating generating layer 606 is thus a heat generating medium. Finally, at the bottom most layer of the internal layer arrangements 602-610 of FIG. 6 is the heat insulating layer 610 (being formed as an integral layer), which is disposed underneath the second busbar layer 608. The heat insulating layer 610 is arranged to seal and prevent the heat collectively generated by the first busbar layer 604, heat generating layer 606, and second busbar layer 608 from further propagating inwardly of the glove former shell assembly. That is, the heat insulating layer 610 is configured to encourage outwardly, or vertical, propagation of the generated heat towards the outer layer 602 for curing the elastomer formed thereon with the heat. As a result, the outwardly propagating heat becomes more efficiently used, as compared to energy losses through horizontally directed heat transfer flow.

Further, it is to be appreciated that the heat insulting layer 610 may alternatively be in the form of a coating, rather than being formed as an integral layer.

Advantages of the internal layer arrangements 602-610 of FIG. 6 include encouraging emanation and propagation of heat generated (by the first busbar layer 604, heat generating layer 606, and second busbar layer 608) outwardly and perpendicularly from, rather than along the plane of the glove former shell assembly to ensure a substantially even distribution of the generated heat for curing the elastomer formed on the outer layer 602 of the glove former shell assembly. It is to be appreciated that the definition of along the plane of the glove former shell assembly means that the heat propagates in a direction along the surface of the glove former shell assembly. In addition, the internal layer arrangements 602-610 of FIG. 6 has simplicity in terms of ease in fabricating the arrangements 602-610, and thus will be cost effective. Moreover, the internal layer arrangements 602-610 of FIG. 6 is geometry and dimension independent, in that irrespective of the simplicity/complexity of the shape/geometry of the glove former shell assembly, the heat generated can still be substantially distributed in an even manner across the glove former shell assembly.

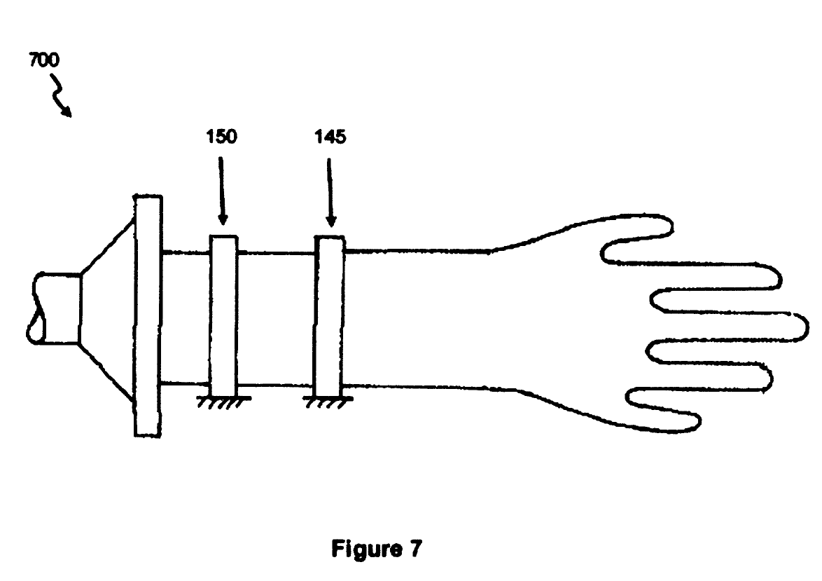

FIG. 7 is a side elevation view of an example glove former shell assembly 700, which incorporates the internal layer arrangements 602-610 of FIG. 6. It will be appreciated that glove former shell assembly 700 of FIG. 7 also incorporates a pair of polarity rings 145, 150, which are similar to that as afore described for FIG. 5, and hence not repeated for brevity. In this instance, the polarity rings 145, 150 are respectively electrically configured with positive and negative polarities (i.e. “+VE” and “−VE”), but will be appreciated that the reverse configuration is also possible, depending on requirements of an intended application.

FIG. 8 is a side view of another internal layer arrangements 602-606, 802, 610 of a glove former shell assembly, according to yet a further embodiment of the present invention. In this instance, internal layer arrangements 602-606, 802, 610 of FIG. 8 is largely similar to the internal layer arrangements 602-610 of FIG. 6, except that the second busbar layer 608 of FIG. 6 is now replaced by a variant second busbar layer 802 of FIG. 8. Specifically, instead of being an integral layer, the variant second busbar layer 802 is formed using an electrically conductive coating, such as for example silver conductive coating. The variant second busbar layer 802 is also electrically arranged with a negative polarity (i.e. “−VE”). It is to be appreciated that the internal layer arrangements 602-606, 802, 610 of FIG. 8 can also be used by the glove former shell assembly 700 of FIG. 7. This internal layer arrangements 602-606, 802, 610 of FIG. 8 is advantageous in that it provides a greater flexibility to a geometry and complexity that may be adopted for a glove former shell assembly, which is especially useful for glove former shell assemblies arranged to produce surgical gloves, household gloves, or the like which have complex shapes.

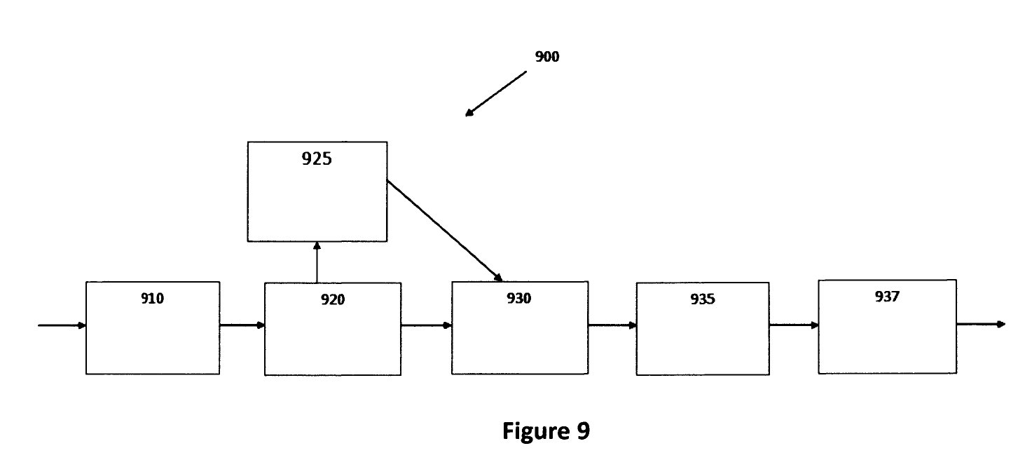

FIG. 9 shows a method of manufacturing 900 according to one embodiment of the present invention. The method 900 includes the steps of:
forming a thermally conductive outer layer 910 in the shape of a dip product, said outer layer having an inner surface, and an outer surface arranged to receive a film of elastomer;
forming an electrically conductive layer 920 on said inner surface;
placing a pair of resistive patterned tracks 930 on said electrically conductive layer;
connecting 935 said pair of resistive patterned tracks to an electrical power supply to provide electrical communication to the said resistive patterned tracks so as to heat said former assembly for curing said elastomer; and
electrically connecting 937 said pair via an intermediate portion of said electrically conducting layer and, generating resistive heat from said intermediate portion.

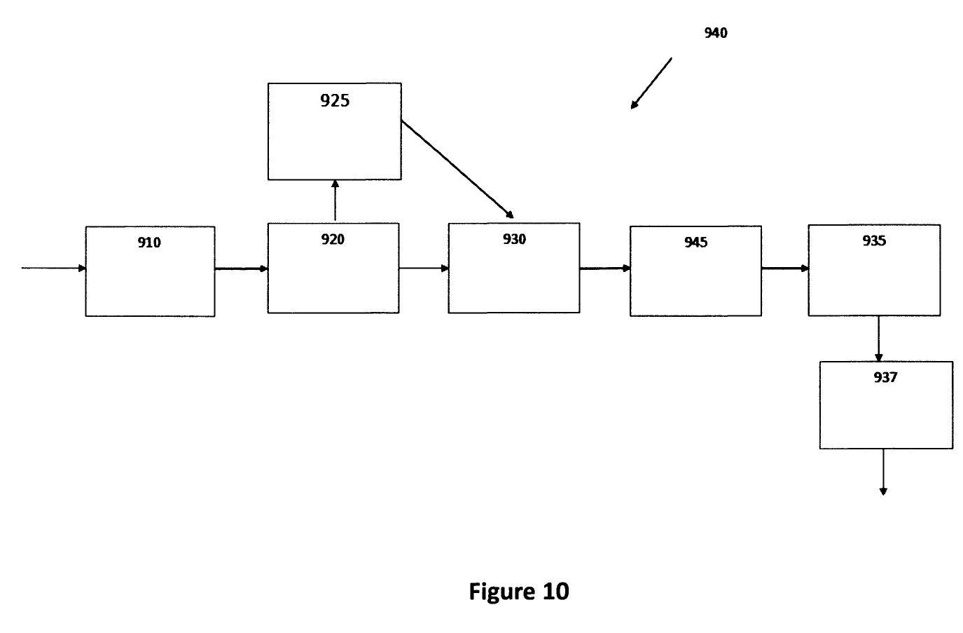

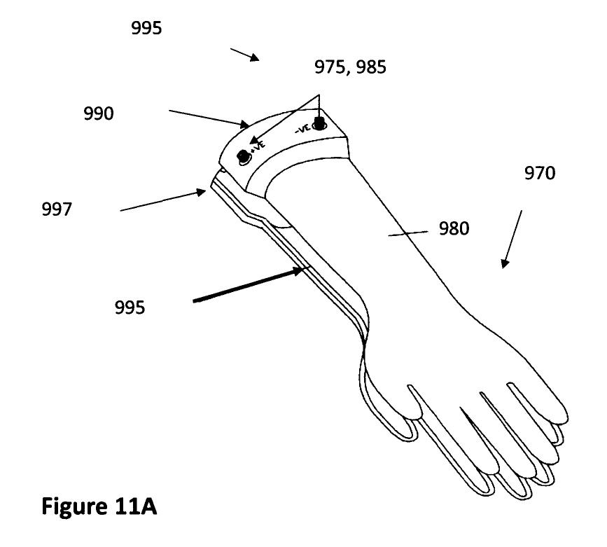

FIG. 10 shows a method of manufacturing 940 according to an alternative embodiment of the present invention. In this embodiment, the method 940 may include the steps of:
forming a thermally conductive outer layer 910 in the shape of a dip product, said outer layer having an inner surface, and an outer surface arranged to receive a film of elastomer;
forming an electrically conductive layer 920 on said inner surface;
placing a pair of resistive patterned tracks 930 on said electrically conductive layer;
varying 945 one or more dimensions of said pair of resistive patterned tracks at pre-determined areas of said outer layer, and so varying a distribution of heat about said electrically conductive layer;
connecting 935 said pair of resistive patterned tracks to an electrical power supply to provide electrical communication to the said resistive patterned tracks so as to heat said former assembly for curing said elastomer; and
electrically connecting 937 said pair via an intermediate portion of said electrically conducting layer and, generating resistive heat from said intermediate portion.

In a further embodiment, the methods of manufacturing 900, 940 may include the step of trimming and disposing 925 of the electrically conductive layer or areas formed in excess on the outer layer. In this embodiment, a multi-axis machining equipment may be provided for this purpose. The multi-axis machining equipment may be a standard device such as a multi-axis CNC (Computer Numerical Cutting) router or a laser engraving head, for instance, a device used for fibre laser marking. Such a laser engraving head may be programme to form the pre-determined shapes required for the specific patterns parallel to the required resistive track patterns within the thermally conductive shell. This step 925 may take place prior to placing 930 the pair of resistive patterned tracks on the electrical conductive layer.

According to one embodiment of the present invention, the thermally conducting layer may be an electrically conductive polymer. In this embodiment, the thermally conducting materials may include any one or a combination of thermal conducting materials such as modified nylon.

In an alternative embodiment, the thickness of the thermally conductive outer layer may be within the range of 1.5 to 4 mm. It may be appreciated that the thickness of the outer layer may vary according to the structural and strength requirements of an intended application.

In other embodiments, the thermal conductivity range of the thermally conductive outer layer or shell is within the range 2 to 15 W·m⁻¹·K⁻¹.

In any of the various embodiments, the pair of resistive patterned tracks may be electrically conductive busbar layers.

In a further embodiment, the pair of resistive patterned tracks may be formed using electro-deposition.

According to one embodiment, the electrically conductive layer may include any one or a combination of the following electrically conductive materials: silver, nickel, copper, aluminium, zinc and graphite. In addition, it may be appreciated that the thickness of the electrically conductive layer applied or formed on the outer layer may vary according to the type of material employed and further, the electrical conductivity requirements of an intended application.

In a further embodiment, the electrically conductive layer may be a mixture in paste form. Alternatively, the electrically conductive layer may be in solution form which makes it suitable for spray techniques commonly employed in the manufacture of former assemblies.

In an alternative embodiment, the thermally conductive outer layer is formed using injection molding.

In another embodiment, the electrically conductive layer may be formed using spraying techniques. In the case of an electrically conductive layer, this may be formed using a thermal spraying method, for instance, an arc wire spray method, and so a controlled electrical conductivity and resistivity balance may be required. For example, this may be achieved using a Nichrome (such as in the alloy 80% nickel, 20% chromium) based material, to act as the heater element layer and patterned accordingly to form the resistive tracks later.

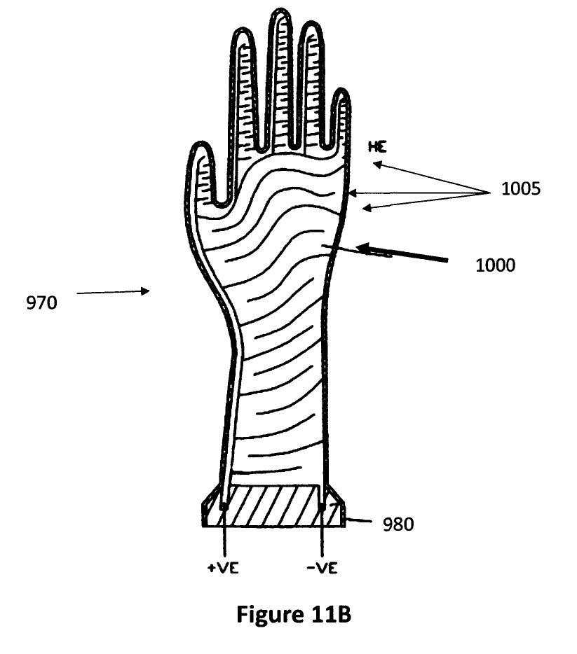

FIG. 11A shows a disassembled glove former shell assembly manufactured according to the method of manufacturing 900. Here, the assembly 970 includes a thermally conductive outer layer 980 for receiving the elastomeric film. In this embodiment, electrical connection between the electrical power supply and the pair of resistive patterned tracks may be provided via respective positive and negative mechanical buds 975, 985 at an end proximate 995 to the fingertips of the glove former shell assembly. The mechanical buds may include any one or a combination of the following materials with good electrical conductivity: copper, aluminium and brass.

Similar to the embodiments discussed in the preceding paragraphs, the outer layer may comprise an outer shell in two halves 990, 997. Further, the two halves may be permanently sealed, such as through heat sealing or vibrational welding or releasably engaged to provide maintenance to the internal heating medium.

The pair of resistive patterned tracks 1005 placed on the electrically conductive layer 1000 of a glove former shell assembly 970 may be more clearly seen in FIG. 11B.

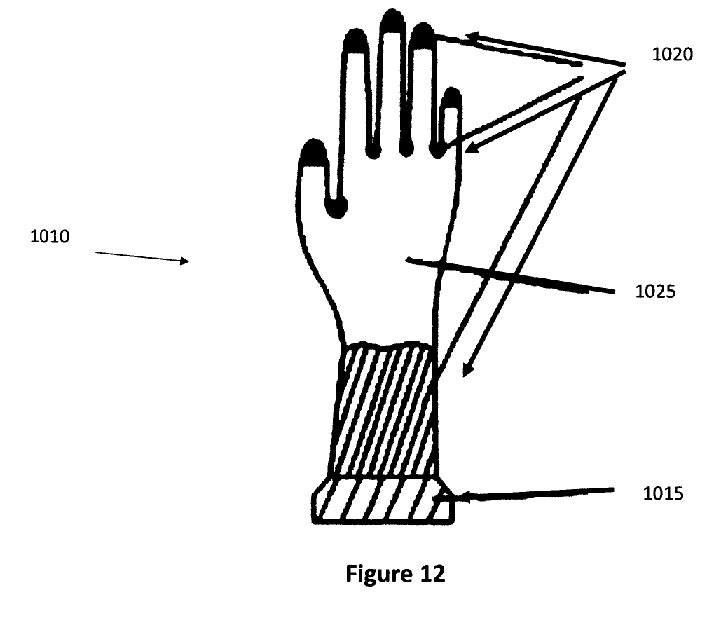

FIG. 12 is a heat distribution profile of a glove former shell assembly 1010 according to a further embodiment of the present invention. In this embodiment, the complexity of the shape and/or geometry of the glove former shell assembly 1010 requires that the cuff area 1015 and the areas at or proximate to the back of the palm 1025 (collectively called “pre-determined or targeted area”) receive little or no heat. In contrast, the area at or proximate to the finger tips and in between fingers (collectively called “pre-determined or targeted area”) requires heat at a higher intensity for curing the elastomer. As discussed in the preceding paragraphs, the heat intensity and distribution may be provided at the pre-determined areas in this case by varying the dimensions of the resistive patterned tracks placed at the on the electrically conductive layer. For instance, resistive patterned tracks may not be placed in areas at or proximate to the cuff 1015 and palm 1025. On the contrary, a pair of resistive patterned tracks of significant thickness may be placed at areas at or proximate to the finger tips to enhance the heat intensity and distribution provided.

### INDUSTRIAL APPLICABILITY

The dipping form of the present invention finds ready industrial application in the glove making industry. With suitable modifications it is also suitable for the condom and balloon making industries and other industries where thin elastic material are made that require heating for drying and curing.

Those skilled in the art will appreciate that various modifications may be made to the present invention without departing from the inventive concept behind the invention. The embodiments of the invention described herein are only meant to facilitate understanding of the invention and should not be construed as limiting the invention to those embodiments only. Those skilled in the art will appreciate that the embodiments of the invention described herein are susceptible to variations and modifications other than those specifically described. It is to be understood that the invention includes all such variations and modifications that fall within the scope of the inventive concept behind the invention.

1-12. (canceled)

13. A method of manufacturing a former assembly comprising the steps of:
forming a thermally conductive outer layer in the shape of a dip product, said outer layer having an inner surface, and an outer surface arranged to receive a film of elastomer;
forming an electrically conductive layer on said inner surface;
placing a pair of resistive patterned tracks on said electrically conductive layer;
connecting said pair of resistive patterned tracks to an electrical power supply;
electrically connecting said pair via an intermediate portion of said electrically conducting layer and, generating resistive heat from said intermediate portion for curing said elastomer.

14. The method according to claim 13, wherein said pair of resistive patterned tracks are electrically conductive busbar layers.

15. The method according to claim 13, wherein the placing step further includes the steps of varying one or more dimensions of said pair of resistive patterned tracks at pre-determined areas of said outer layer, and so varying a distribution of heat about said electrically conductive layer.

16. The method according to claim 14, wherein the placing step further includes the steps of varying one or more dimensions of said pair of resistive patterned tracks at pre-determined areas of said outer layer, and so varying a distribution of heat about said electrically conductive layer.

17. The method according to claim 13, wherein said resistive patterned tracks are formed using electro-deposition.

18. The method according to claim 14, wherein said resistive patterned tracks are formed using electro-deposition.

19. The method according to claim 15, wherein said resistive patterned tracks are formed using electro-deposition.

20. The method according to claim 16, wherein said resistive patterned tracks are formed using electro-deposition.

21. The method according to claim 13, wherein said thermally conductive outer layer is formed using injection molding.

22. The method according to claim 14, wherein said thermally conductive outer layer is formed using injection molding.

23. The method according to claim 15, wherein said thermally conductive outer layer is formed using injection molding.

24. The method according to claim 16, wherein said thermally conductive outer layer is formed using injection molding.

25. The method according to claim 17, wherein said thermally conductive outer layer is formed using injection molding.

26. The method according to claim 18, wherein said thermally conductive outer layer is formed using injection molding.

27. The method according to claim 19, wherein said thermally conductive outer layer is formed using injection molding.

28. The method according to claim 13, wherein said electrically conductive layer is formed using thermal spraying techniques.

29. The method according to claim 14, wherein said electrically conductive layer is formed using thermal spraying techniques.

30. The method according to claim 15, wherein said electrically conductive layer is formed using thermal spraying techniques.

31. The method according to claim 17, wherein said electrically conductive layer is formed using thermal spraying techniques.

32. The method according to claim 21, wherein said electrically conductive layer is formed using thermal spraying techniques.
